(19) United States
(12) Patent Application Publication
(10) Pub. No.: US 2026/0199260 A1
(43) Pub. Date: Jul. 16, 2026

(54) PATIENT TREATMENTS WITH INFUSED WOUND-HEALING MATERIALS

(71) Applicant: Sheila Nazarian, Los Angeles, CA (US)

(72) Inventor: Sheila Nazarian, Los Angeles, CA (US)

(21) Appl. No.: 19/442,233

(22) Filed: Jan. 7, 2026

Related U.S. Application Data
(60) Provisional application No. 63/743,814, filed on Jan. 10, 2025.

(51) Int. Cl. A61K 9/70; A61K 33/38; A61K 36/28; A61K 38/48

(52) U.S. Cl. CPC A61K 9/7084; A61K 33/38; A61K 36/28; A61K 38/4873

(57) ABSTRACT

Example patient treatments and methods of designing, making and using the same are directed to a carrier material configured to be placed over a skin of a patient. The patient treatment also includes an infused material comprising a skin treatment component. The infused material is exposed such that the skin treatment component is in contact with the skin of the patient when the carrier material is placed over the skin of the patient. Additionally, the skin treatment component reduces a swelling of the patient and reduces bruising of the patient.

### CROSS-REFERENCE TO RELATED APPLICATIONS

This application claims the benefit of U.S. Provisional Patent Application No. 63/743,814, entitled “Patient Treatments with Infused Wound-Healing Materials”, filed Jan. 10, 2025, the disclosure of which is hereby incorporated by reference herein in its entirety for all purposes.

### BACKGROUND

Temporary patient treatments such as patches, sheet masks, bandages, dressings, tapes, strips, and sheets are applied to a wound (e.g., including incisions created intentionally during a surgical procedure and other wound types) or skin surface (e.g., to provide treatments) for a variety of medical purposes. For example, a temporary patient treatment may be applied in a controlled manner to seal an incision after a surgical procedure, to cover an area of skin that has been treated in some manner (e.g., a sheet mask), or for some other at-home use. Example temporary patient treatments may include elastic materials such as polymers, silicone, or the like, and may come in a variety of forms to accommodate different wound sizes and shapes. The general principle of many temporary patient treatments is to fix the treatment to multiple locations on adjacent tissue (e.g., skin), such as by an adhesive material on a lower surface of the elastic material of the patient treatment. The elastic material will naturally seek to compress, applying lateral pressure to the wound in a manner that closes the wound and creates a likelihood of adjacent portions of skin previously separated by the wound coming into contact. The compressive force and contacting surfaces aid in healing of the wound, and by covering the wound the patient treatment reduces the likelihood of infection throughout the healing process. Despite existing temporary patient treatments in closing and/or covering the wound or skin surface and protecting the wound or skin surface from external materials, the longer it takes for a wound or skin surface to heal, the higher the likelihood of infection, potential dislodging of the temporary patient treatment, or of the user ceasing use of the temporary patient treatment or failing to follow instructions. As a result, existing temporary patient treatments often require excessive patient monitoring and instructions, and often fail to function adequately in effectively healing the wound or skin surface, resulting in avoidable return visits by patients or adverse patient responses.

### SUMMARY

In at least some example approaches, a patient treatment is provided having infused skin treatment materials. The patient treatment comprises a carrier material configured to be placed over a skin of a patient. The patient treatment also includes an infused material comprising a skin treatment component. The infused material is exposed such that the skin treatment component is in contact with the skin of the patient when the carrier material is placed over the skin of the patient. Additionally, the skin treatment component reduces a swelling of the patient and reduces bruising of the patient.

In some example approaches, a patient treatment comprises a carrier material configured to be placed against skin of a patient. In at least some of these examples, the carrier material comprises at least one of silicone, Silagen®, a sheet mask, a polymer, a band-aid, a wound dressing, a gel, a steri-strip, SYLKE®, or Mepelex. Th patient treatment further comprises an infused material disposed on or within at least a portion of the carrier material. The infused material may include a skin-treatment component including arnica, bromelain, and silver. The infused material may be exposed at a surface of the carrier material such that, when the carrier material is placed against the skin of the patient, the infused material contacts the skin. The carrier material may be one of a patch having the infused material as a portion of the patch, a sheet mask having the infused material as a coating applied to the sheet mask, or a carrier having the infused material as an insert positioned to contact the skin when the carrier material is placed on the patient. The carrier material further comprises an adhesive portion that adheres to the skin, the adhesive defining a pattern of perforations between the adhesive portion and the infused material that provides elasticity of the patient treatment.

In at least some example illustrations, a method of making a patient-treatment article comprises providing a blank of carrier material configured to be placed over skin of a patient. The method also includes infusing a skin-treatment component material into at least a portion of the carrier material. The skin-treatment component material is exposed at a surface of the carrier material such that the infused material is configured to contact a patient skin when the article is applied.

### BRIEF DESCRIPTIONS OF THE DRAWINGS

The above and other features of the present disclosure, its nature, and various advantages will be more apparent upon consideration of the following detailed description, taken in conjunction with the accompanying drawings in which:

FIG. 1A depicts an exemplary wound patch with infused materials in accordance with an embodiment of the present disclosure;

FIG. 1B depicts an exemplary sheet mask with infused materials in accordance with an embodiment of the present disclosure;

FIG. 2 depicts an exemplary wound patch having regions of varied thickness in accordance with an embodiment of the present disclosure;

FIG. 3 depicts an exemplary wound patch having perforations within elastic extendable portions of the wound patch in accordance with an embodiment of the present disclosure;

FIGS. 4A-4G depict additional exemplary temporary patient treatment configurations in accordance with embodiments of the present disclosure;

FIG. 5 depicts steps of designing an infused wound healing patch in accordance with embodiments of the present disclosure;

FIG. 6 depicts steps of manufacturing an infused wound healing patch in accordance with embodiments of the present disclosure; and

FIG. 7 depicts steps of applying an infused wound healing patch in accordance with embodiments of the present disclosure.

### DETAILED DESCRIPTION

Patient treatments in accordance with the present disclosure may be implemented in a variety of suitable coverings (e.g., silicone, Silage®, a sheet mask, a polymer, a band-aid, a wound dressing, a gel, a steri-strip, SYLKE®, Mepelex, an implant, etc.) or devices (e.g., an injection/tube) to deliver effective dosages and concentration combinations of active substances directly to impacted patient skin and/or wounds (e.g., as a result of surgeries, skin treatments, skin injury, burns, abrasions, or trauma, etc.). Examples of conditions with which the present disclosure can assist in expediting patient healing and improved comfort include, but are not limited to, cuts, incisions, abrasion, skin roughening, chemical treatments, laser treatments, surgical treatments, bruising, burns, topical cancers, non-cancerous or precancerous lesions.

In some embodiments, a patient treatment includes a combination of multiple skin healing materials infused and/or coating a carrier material such as a wound patch, sheet mask, bandage or the like. The materials of the infusion and/or coating include at least arnica and bromelain, and are exposed to the target area such that the arnica and the bromelain-infused carrier material is placed over the patient's skin. The combination of materials can, for example, reduce both swelling (due to the effects of the arnica) and bruising (due to the effects of the bromelain). In some examples, additional treatment materials may be added. For example, the addition of silver to the carrier material provides an anti-microbial impact to the patient's skin.

The patient treatment can include varied concentrations of active ingredients (e.g., bromelain, arnica, and silver) depending on the particular purpose and application of the patient treatment. Certain types of treatments may be more likely to result in bruising, while other types of treatments may be more likely to cause swelling. Accordingly, the absolute and relative concentrations of bromelain and arnica may be adjusted depending on the application, either in manufacturing or preparation of the patient treatment. Similarly, concentrations of other ingredients such as silver can be modified for particular applications of the patent treatment where anti-microbial effects are required.

The patient treatment is provided in varied configurations depending on the particular purpose and application of the patient treatment. Some applications require additional compressive forces or elasticity, which can be achieved with adhesives, perforations, variations in shape, and other modifications as described herein. The active materials can be applied as part of the adhesive, as a coating, as an infusion, and combinations thereof to deliver the active ingredients to the necessary wound location.

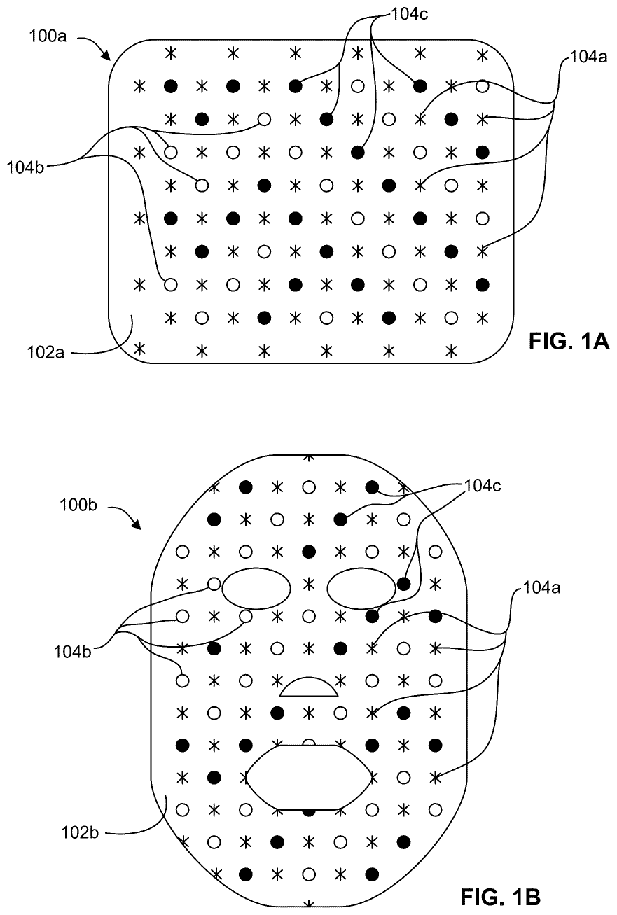

FIGS. 1A and 1B depict exemplary wound patches 100 with infused materials in accordance with embodiments of the present disclosure. More specifically, FIG. 1A illustrated a generally rectangular-shaped wound patch 100a, while FIG. 1B illustrates a face-shaped wound patch or face mask 100b. It will be understood that the particular wound patch shape, configuration, and concentrations and locations of infused materials are provided as examples only.

In the example illustrated in FIG. 1A, a wound patch 100a is configured to be placed on an area of skin (not shown in FIG. 1A) that has been treated in some manner. Areas necessitating treatment may include an incision, a cut from an accident, a burn, or a non-invasive skin treatment. An incision could have been made in the skin during a surgical procedure. An accidental cut, abrasion, or burn could have been caused by an accident, or the skin could also have been “compromised” in some fashion, i.e. a non-invasive cosmetic procedure, including but not limited to a laser treatment, a chemical peel, a microdermabrasion, or micro-needling. The wound patch 100a can increase the rate of healing of the wound, protect the wound from exposure to the elements which increase a likelihood of infection, and/or provide materials to be infused into the affected area.

FIG. 1A shows the underside of wound patch 100a, i.e., which may be applied to an area of skin. The wound patch 100a comprises a carrier material 102 that is infused with materials such as silver, arnica and bromelain in areas 104 thereof. In FIG. 1A, silver is infused in areas 104a illustrated as asterisks, arnica is infused in areas 104b illustrated as empty circles, and bromelain is infused in areas 104c illustrated as shaded/filled-in circles. Although these materials are shown to be infused into a carrier material in a regular pattern, the materials can be infused into the patch (or portions thereof, including only portions of the length, width, or thickness) in appropriate patterns for particular end-use applications and/or be coated onto the wound patch (e.g., within an adhesive or a surface gel). Additionally, materials other than silver, arnica, and bromelain can be used, depending on the reason for use of the wound patch. In FIG. 1A, the silver, arnica, and bromelain are used because they assist with different aspects of healing and/or infection prevention. For example, silver is antimicrobial making it a good component to add when trying to prevent infection. Arnica, which is derived from plants in the sunflower family, is useful for the prevention of swelling. This swelling can be caused by inflammation of small vessels in tissue. Bromelain, a proteolytic enzyme found in pineapple juice and stems, is anti-inflammatory and used to prevent inflammation. Bromelain helps decrease bruising by breaking down proteins that trap fluids in the tissues after an injury or trauma. The combination of some or all of these materials and their direct application to wound locations allows them to interact collectively to improve patient recovery and response to wounds.

In an example, the carrier material 102 may include or be entirely comprised of silicone, although other materials may be utilized as described herein. The silicone carrier material may generally provide no or minimal healing benefit to the affected area on its own. The silicone is used to enable the active materials that are infused in the silicone (silver, arnica and bromelain in FIGS. 1A and 1B) to permeate or infuse into the affected area of a patient.

The wound patch 100a can be applied to the affected area at different stages throughout the healing process. For example, the wound patch 100a may be applied immediately after a procedure. In this application, the wound patch 100a is used primarily to assist with healing and/or to prevent infection. Alternatively or in addition, a wound patch 100a might be applied at a later stage once healing has begun or is almost complete. This latter wound patch might contain different active ingredients. For example, vitamin E might be infused in the wound patch 100a to prevent scarring. Moreover, multiple wound patches 100 can be used over time on the affected area as needed. Each patch 100 can have different active ingredients, configurations, shapes, adhesives, and/or thicknesses depending on the desired application and results.

In accordance with the present disclosure, and for any embodiments described herein, the treatment materials may be infused and/or coated in a variety of respective manners and concentrations for different purposes. For example, different patches may have different respective concentrations of arnica, bromelain, and silver depending on the different skin conditions to be treated and their respective need for swelling reduction, bruise prevention, and antimicrobial protection. Moreover, a single carrier material 102 of a wound patch 100a may have different concentrations at different locations, for example, relative to an incision, abrasion, or other impacted area of a wound.

In one embodiment the wound patch 100a is primarily a silicon material or layer 102, in which the various materials are infused into it. In the example illustrated in FIG. 1A, silver, arnica, and bromelain are infused into the silicon material 102. Additional compounds or materials can be infused into the material or layer 102 to allows for the permeation or infusion of the active materials, e.g., compounds, into an affected area. Some other possible carrier materials can include silicone, Silagen®, a polymer, gels or foams (e.g. Mepilex®).

In an embodiment, the wound patch 100a also has an adhesive (not specifically depicted in FIG. 1A) over a portion of it that allows the wound patch 100a to be securely placed over the desired area. The adhesive can be its own separate layer or can be included within the carrier layer 102 (in FIG. 1A, a silicon material). The adhesive can be any material(s) which will secure the wound patch 100a to the affected area. Adhesives such as acrylic-based adhesives, silicon-based adhesives, rubber-based adhesives, hydrocolloid adhesives, hydrogel-based adhesives, pressure sensitive adhesives, and/or bioadhesives can be used. In some embodiments, some or all of the treatment materials can be infused into the adhesive. The top side of the wound patch may also be airtight and/or watertight. This would prevent any air and/or moisture from entering the wound or cut area, while also preventing infused materials from escaping from the wound patch 100a. In embodiments where some or all of the adhesive does not include any active materials, the adhesive can be located or patterned in a manner to avoid wound and/or incision site(s).

There are many types of active materials that can be used in the wound patch 100a to achieve various desired effects. Active materials that can protect skin from the sun, are anti-aging, repair sun and age damage, repair acne, prevent acne or lighten dark spots can be used. Any active material that can be infused into the skin can be used in the wound patch 100a. The diverse treatments necessary to remedy the affected area require that different wound patches contain different active materials.

As noted above, if it is desired to treat an incision in a skin area from a surgery, or a cut, abrasion or burn from an accident, an example wound patch 100a may include more active materials to reduce inflammation, resist infections, and break down bruises. In this wound patch it may be desired to have materials such as silver, arnica, bromelain, Silvadene (silver sulfadiazine), MediHoney, or other antibiotics.

In another example, if the patient/user had a non-invasive cosmetic procedure including but not limited to a laser treatment, a chemical peel, a microdermabrasion, or micro-needling, other materials might be desired. For example, to protect skin which may have become vulnerable to the sun due to a procedure, the mask may be infused with sunscreen as noted above. Merely as examples, sunscreens may include titanium oxide, silicon oxide, or zinc oxide. In addition, after these types of procedures (i.e., that typically cause vulnerability to sunlight), the skin is more readily receptive to medications and/or nutrients that might help the skin. For example, the wound patch 100a might be infused with anti-aging and acne-repair or acne-prevention materials. Some of these active materials may include vitamin C, retinol, vitamin A derivatives (retinol) to help repair the skin from fine lines and wrinkles and to repair acne damage and vitamin E. The wound patch could also be infused with growth factors such collagen and elastin to help the skin repair itself. The wound patch 100a could also be infused with skin brightening agents such as kojic acid and hydroquinone to minimize the appearance of dark spots on one's skin. These materials can be infused in the carrier material or can be dipped in another carrier material and applied to the surface of skin.

The wound patch 100a can also include exosomes. These are small membrane-bound vesicles that play a role in extracellular communication by telling cells how to behave. More specifically, they communicate by carrying proteins, lipids, and genetic material between cells. Exosomes can be in a powder form and can be easily sprinkled throughout the silicone or any other compatible carrier material. Any combination of active materials can be infused in the carrier material.

Referring now to FIG. 1B, an exemplary sheet mask 100b is illustrated with infused and/or coated treatment materials in accordance with an embodiment of the present disclosure. The sheet mask 100b depicted in FIG. 1B provides just one example of an alternative application method of providing treatment materials via a carrier material 102b as infusions or coatings. The sheet mask 100b, in some examples, may be identical to wound patch 100a, apart from the face-shaped configuration illustrated in FIG. 1B. FIG. 1B shows that the sheet mask 100b is infused with silver, arnica and bromelain in areas 104a, 104b, 104c thereof, respectively (where silver is represented by asterisks, Arnica is represented by empty circles, and bromelain is represented by shaded circles). Although these materials are shown to be infused and/or coated on the sheet mask in a regular pattern, the materials can be infused and or coated on the sheet mask 100b in any pattern and/or be randomly placed on the sheet mask 100b. Additionally, other materials can be infused or otherwise used depending on the reason for the use of the mask 100b.

Although FIG. 1B shows a face mask (i.e., shaped to fit a patient's face), wound patches 100 can be formed into any desired shape to fit other skin areas and anatomical locations. As in FIG. 1A, materials can be infused into or coated onto the carrier material 102b of the sheet mask 100b that allows for the infusion of the active materials into the affected area. Some other possible carrier materials can include silicone, silage, a polymer, gels or foams (e.g. Mepilex®).

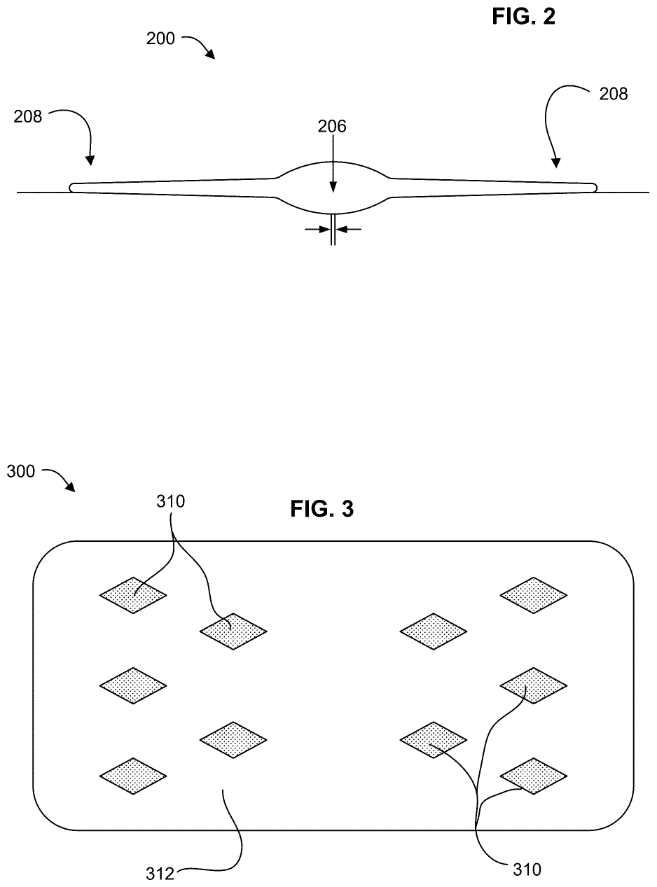

FIG. 2 depicts a side view of an exemplary wound patch 200 having varying thickness in different areas of the wound patch 200, in accordance with an embodiment of the present disclosure. In an example, the wound patch 200 is wound patch 100a or 100b. In the example illustrated in FIG. 2, a center region 206 of the patch 200 is thicker than edge regions 208 of the patch 200. In one embodiment, the thicker the patch 200, the more materials are infused or contained therein. The thicker center region 206 of the patch may be placed over an epicenter of a desired area of a skin area. The patch 200 or portions thereof may be covered with an adhesive material to allow the user to secure the wound patch 200 on the skin. If the elasticity of the carrier 202 is constant throughout the patch, the vertical pressure applied by the center region 206 of the wound patch 200 to the epicenter of the desired area may be relatively greater than that applied by the thinner edge regions 208 of the wound patch 200. The increased pressure can assist with faster healing and also facilitate the desired materials being infused into the desired area, e.g., corresponding to skin area(s) contacting the thicker/center region 206) at an increased rate. It is also possible that the center region 206 of the patch 200 may be infused with different materials than those in the edge regions 208 of the patch. Further, an elasticity of the adhesive material can be adjusted in any manner to achieve desired pressures or lack of pressure on specific areas of the desired area (i.e., the wound) and to adjust to different desired vertical and/or lateral pressures. Different types of adhesives can also be used within the wound patch 200 if desired.

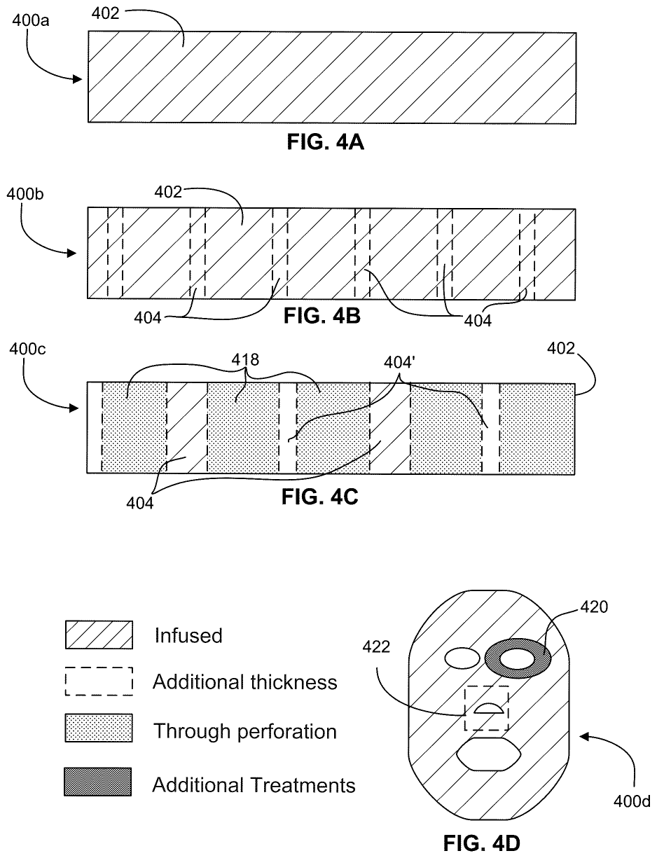

FIG. 3 depicts an exemplary wound patch 300 having perforations 310 within one or more elastic or extendable portions 312 of the wound patch in accordance with an embodiment of the present disclosure. FIG. 3 shows the perforations 310 as being diamond-shaped in the elastic regions 312 which extend with adhesive application or respective portion of the patch to the skin surface. These perforations 310 allow the elastic regions 312 of the patch 300 to expand more easily and assert different types and amounts of pressure on a wound and surrounding areas. For example, the perforations 310 in the adhesive may generally allow stretching of elastic portion 312 as skin to which the wound patch 300 is adhered stretches during bending of a patient/user's body, or the like. The size, shape, number of perforations 310, density and coverage of perforations 310 can be adjusted based on the desired result of the wound patch 300. These variables can change the elasticity and expandability of the wound patch 300. By doing so, the user is able to adjust a direction and/or amount of pressure on an affected area, and further increase or decrease a breathability of the wound patch 300 to desired level(s).

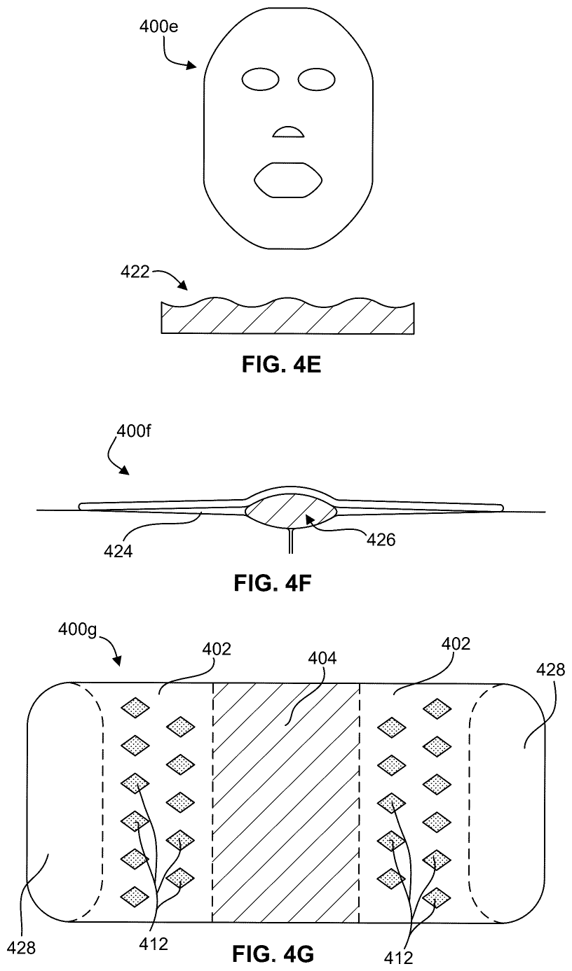

FIGS. 4A-4G depict plan views of additional exemplary configurations of a wound patch 400, in accordance with embodiments of the present disclosure.

FIG. 4A depicts an exemplary wound patch 400a that is an infusible cuttable sheet 402. The sheet 402 can be cut into a desired size for use on a patient. In this embodiment, the cuttable sheet 402 can be infused and/or coated with one or multiple treatment materials. These treatment materials can be evenly distributed spatially throughout the sheet 402 or can be distributed spatially in a desired pattern, e.g., with an increased concentration of treatment materials in one or more regions of the wound patch 400a. Further, the materials can be consistent concentrations in regions of the wound patch 400a where present, or can vary depending on the location or the material. Also, the materials can be used in the same or different amounts and the amounts used can also be evenly distributed spatially or randomly distributed spatially.

FIG. 4B depicts an exemplary wound patch 400b that is an infused cuttable sheet 402 with thickened regions 404 for attachment and wound coverage. The thickened regions 404 may define a relatively greater thickness than adjacent areas of the cuttable sheet 402. The thicker regions 404 can be evenly placed along the sheet or can be placed in patterns. Further, there can be multiple thicknesses or thicker regions 404 within the cuttable sheet 402 which can also be placed in a regular or distributed pattern. These thicker regions 404 may contain a higher or lower concentration of infused materials, no infused materials, a higher or lower amount of infused materials, or different materials in different areas depending on the desired results. The wound patch 400b can be provided with or without an adhesive. If no adhesive is provided, a user may create a desired pattern in the sheet 402 (such as by cutting), place the wound patch 400b over the desired area, and then secure an adhesive, bandage, or the like on top of the wound patch 400b, thereby securing the wound patch 400b in place. If a consistent apparatus/method is used to secure the wound patch 400b, a vertical pressure on the thickened portions 400 of the wound patch 400b may be relatively greater than a vertical pressure on the thinner portions of the patch 400b (i.e., in between thickened regions 404).

FIG. 4C depicts an exemplary wound patch 400c that is an infused cuttable sheet 402 with interspersed thicker regions 404 with infusions and/or coatings, perforated regions 418, and thicker regions 404′ without infusions and/or coatings. In this example embodiment, infusions and/or coatings are limited to specific regions for application to the patient (e.g., within a thickened region 404 to apply additional pressure), and have, on each side, a perforated region 418 and two non-infused thicker regions 404′. With such a sheet 402, the wound patch 400c may be cut based on size of the region of application, desired pressure application, and the like. For example, with the non-infused thicker regions 404′ used for placement of the respective edges of a wound patch 400c cut from the cuttable sheet 402.

FIG. 4D depicts an exemplary wound patch 400d that is an infused and/or coated sheet mask in accordance with an embodiment of the present disclosure. The sheet mask 400d depicted in FIG. 4D can be infused and/or coated with any number of materials depending on the desired results as previously discussed. These materials can be placed throughout the mask 400d in specific locations as desired. For example, if a patient received treatment around an eye area 420, additional moisturizers, anti-aging materials (such as retinol, vitamin C, or vitamin A, collagen, elastin) can be infused in the wound patch 400d in the eye area 420, thereby facilitating placement of the additional infused materials around the eye area. In another example, if an anti-acne treatment was done, it may be desired to place a wound patch containing anti-acne materials around the nose, chin and/or forehead area, which may be implemented with a thicker infused area 422. In yet another example, patches infused with skin-whitening materials (such as kojic acid and hydroquinone) may be placed in specific areas more prone to dark spots. Further, if a wound or incision on the face is being treated, the mask 400d may contain specific materials to prevent infection, inflammation, or scarring. This sheet mask 400d could be made specifically for a unique patient's needs, or remain more generic depending on the procedure to which the wound patch 400d is targeted.

FIG. 4E depicts an exemplary wound patch 400e that is an infused sheet mask with a bath 422, in accordance with an embodiment of the present disclosure. The wound patch 400e may be wound patch 400d, in an example. In the embodiment of FIG. 4E, the sheet mask 400e functions as a carrier material while the treatment materials are infused within a gel or aqueous bath 422. During treatment, the sheet mask 400e is used to transfer the infused treatment materials emanating or evaporating from the bath 422 to the surface of the skin (e.g., the face as in the sheet mask 400e of FIG. 4E, or other suitable skin locations using other sheet mask designs). In some embodiments, the wound patch 400e includes a lower concentration of infused materials, to facilitate continued application after the gel or aqueous bath 422 is applied and absorbed.

FIG. 4F depicts an exemplary wound patch 400f over a wound healing insert 424 in accordance with an embodiment of the present disclosure. In FIG. 4F, the wound healing insert 424 can be infused with one or more desired materials and may be in a variety of forms or formats such as an enclosed gel, putty, flexible, or cuttable material. In this manner, a desired amount of infused material can be inserted or placed at a particular location and held in place by the patch 400f or other adhesive attachment. For example, the wound healing insert 424 may have a pod 426 for delivering an enclosed gel, putty, or other desired material. The wound patch 400f surrounding the pod 426 can be exclusively adhesive or may contain silicon infused materials (e.g., at lower concentrations remote from a wound) as well.

FIG. 4G depicts a wound patch 400g with thicker edge regions 428 for attachment, a thicker infused region 404 in the middle of the patch, and diamond perforations 412 on each side for elasticity in accordance with an embodiment of the present disclosure. In FIG. 4G, the ends of the wound patch 400g are thicker, and do not contain infused materials. Alternatively, active/infused materials can be infused throughout all regions of the wound patch 400g, such as at varied concentrations. A thickened region 404 positioned in the middle of the patch 400g is thicker relative to the sheet 402 and infused with one or more desired treatment materials. The wound patch 400g can include adhesive, and/or an adhesive or bandage may be placed on top of the wound patch 400g, thereby securing the wound patch 400g to a skin area. The bandage or adhesive may have diamond-shaped perforations 412 which can increase an elasticity of the wound patch 400g, and may enable different types and/or amounts of pressure on a wound and surrounding areas. The size, shape, number of perforations 412, density and coverage of perforations 412 can be adjusted based on the desired results of the wound patch 400g. These variables can change the elasticity and expandability of the wound patch 400g. By changing the elasticity and expandability of the wound patch 400g, the user is able to adjust a direction and amount of pressure on an affected area, while also being able to increase or decrease the desired breathability of the wound patch 400g.

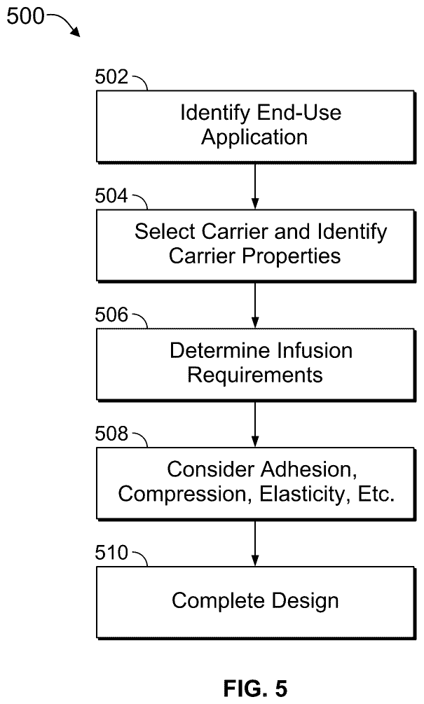

FIG. 5 depicts an example process 500 of designing an infused wound healing patch, in accordance with embodiments of the present disclosure. Although particular steps are depicted in a certain order for FIG. 5, steps may be removed, modified, or substituted, and additional steps may be added in certain embodiments, and in some embodiments, the order of certain steps may be modified. At block 502, an end-use application for a wound patch is determined. As discussed above, end-uses of a wound patch can include incision protection post-surgery or procedure, or the healing of a cut and/or a burn resulting from a non-invasive cosmetic procedure such as a peel or laser treatment, merely as examples. Depending on the end-use desired, the shape, infused materials, concentrations of infused treatment materials, manner of application (infusion and/or coating), and other characteristics of the wound patch can be modified. For example, if the end-use is to treat a particular incision, the wound patch shape can be designed to adequately cover said incision. If the wound patch is to treat a facial procedure, the wound patch may be adapted to the shape of a face mask.

Once the end-use is determined, process 500 may proceed to block 504. At block 504, the wound patch's carrier and desired carrier properties are determined. The carrier may be a silicon base or any other material that allows for infusion of materials and provide a covering over the skin, along with desired properties such as elasticity, etc., as described herein. Once the carrier is determined at block 504, process 500 may proceed to block 506.

At block 506, infusion requirements may be determined. Depending on the desired results, a variety of materials are then infused into the carrier of the wound patch. The wound patch can have any number of materials infused into the carrier. For example, if the wound patch is being used to prevent infection, the carrier of the wound patch might be infused with silver or other available antimicrobials. As previously discussed, there are various types of materials that can be infused into the carrier. Once the specific materials to be infused are determined, the particular concentrations and amounts used of each material are established. Then, based on a particular use of a wound patch, the locations onto which the infused wound patch will be placed can be determined. The infused areas can be placed in a patterned, random or strategic manner. Concentrations of each material may also be selected, for example, with relative concentrations of arnica, bromelain, and silver for different end-use applications.

Proceeding to block 508, the wound patch's adhesion, compression and elasticity may be determined. For example, a location of the adhesive, i.e., whether the adhesive is atop the carrier material or infused into the material, may be determined. Further, at block 508 it may be decided whether the adhesive will be located on the periphery of the wound patch, or be located or distributed evenly or more evenly throughout the entire patch. These are just a few examples of determinations with respect to the type, amount and location of the adhesive used, but this list is nonexhaustive. At block 508, it may also be determined how much and where compression is desired on an affected area being treated with a wound patch. Based on these determinations, the thickness of the wound patch can be adjusted to create greater vertical or lateral pressure in desired locations. For example, for the purposes of treatment, it may be beneficial to create increased/additional vertical pressure directly on the wound and/or incision site so that the carrier materials are infused more readily into that area. In another example, it might be advantageous to apply additional vertical pressure around the wound and/or incision to ensure that the wound patch is secure. In this case, a periphery of the wound patch might have an increased thickness. As noted above, an elasticity of the wound patch may also be determined at block 508. A wound patch might have perforations that can direct the amount of desired pressure or provide ventilation to an affected area. Additional elasticity options can be implemented based on the desired results.

Proceeding to block 510, a design of a wound patch is completed once all the parameters of the wound patch are determined.

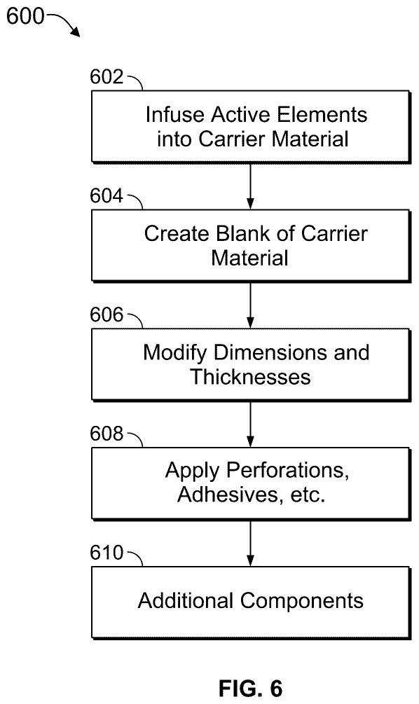

FIG. 6 depicts an example process 600 of manufacturing an infused wound healing patch in accordance with embodiments of the present disclosure. Although particular steps are depicted in a certain order for FIG. 6, steps may be removed, modified, or substituted, and additional steps may be added in certain embodiments, and in some embodiments, the order of certain steps may be modified.

At block 602, active elements are infused into and/or coated onto the chosen carrier material. A number of carrier materials can vary from one to any number depending on the results desired. In addition, concentrations, amounts and locations of one or more active elements are determined. Depending on the design choices, the carrier material can be only a carrier material infused with the desired materials, or the adhesive can also be incorporated into the carrier material as well.

Process 600 may then proceed to block 604, where blanks of the carrier material are created. For example, a thickness of the blank of carrier material may be adjusted. Proceeding to block 606, dimensions and a thickness of the wound patch are determined. At block 608, the desired perforations and adhesives are applied to the wound patch. Finally, at block 610, any additional components desired can be added to the wound patch. These “additional components” may include the printing of various designs, such as logos, use identifiers, or directions.

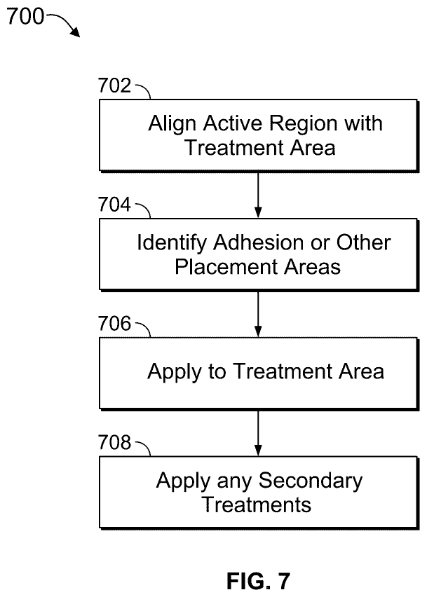

FIG. 7 depicts an example process 700 of applying an infused wound healing patch in accordance with embodiments of the present disclosure. Although particular steps are depicted in a certain order for FIG. 7, steps may be removed, modified, or substituted, and additional steps may be added in certain embodiments, and in some embodiments, the order of certain steps may be modified.

At block 702, the user of the wound patch may align the patch with the treatment area. For example, if the treatment area is an incision, the wound patch may be aligned over the incision. If the treatment area is an entire face area, a sheet mask in the shape of a mask, e.g., wound patch 100b, may be aligned over the face. Once the wound patch is correctly aligned at block 702, the location and type of the adhesion of the wound patch is located in step 704. In some cases, the adhesive can be exposed by peeling off a protective layer. In another model, adhesive can be activated by exposing the patch to moisture or some other material. Once the adhesive is exposed, or properties of the adhesive are otherwise activated, the wound patch may be applied to the treatment area at block 706.

Once the wound patch is applied, process 700 may then proceed to block 708, where any desired secondary treatments can be applied to the area. These secondary treatments can include ultra-violet (UV) light, steam, standard sterilization, and/or activation techniques as are widely understood in the medical field.

The foregoing is merely illustrative of the principles of this disclosure and various modifications may be made by those skilled in the art without departing from the scope of this disclosure. The embodiments described herein are provided for purposes of illustration and not of limitation. Thus, this disclosure is not limited to the explicitly disclosed systems, devices, apparatuses, components, and methods, and instead includes variations to and modifications thereof, which are within the spirit of the attached claims.

The systems, devices, apparatuses, components, and methods described herein may be modified or varied to optimize the systems, devices, apparatuses, components, and methods. Moreover, it will be understood that the systems, devices, apparatuses, components, and methods may have many applications. The disclosed subject matter should not be limited to any single embodiment described herein, but rather should be construed according to the claims.

What is claimed is:

1. A patient treatment with infused skin treatment materials, comprising:
a carrier material configured to be placed over a skin of a patient; and
an infused material comprising a skin treatment component, wherein the infused material is exposed such that the skin treatment component is in contact with the skin of the patient when the carrier material is placed over the skin of the patient, and wherein the skin treatment component reduces a swelling of the patient and reduces bruising of the patient.

2. The patient treatment of claim 1, wherein a first portion of the skin treatment component comprises arnica and a second portion of the skin treatment component comprises bromelain.

3. The patient treatment of claim 2, wherein the infused material further comprises silver configured to provide an anti-microbial impact to the skin of the patient.

4. The patient treatment of claim 1, wherein the infused material comprises a portion of the carrier material.

5. The patient treatment of claim 4, wherein the carrier material comprises a patch.

6. The patient treatment of claim 1, wherein the infused material comprises a coating over a portion of the carrier material.

7. The patient treatment of claim 1, wherein the infused material comprises an insert that is located at the skin of the patient by the carrier material.

8. The patient treatment of claim 1, wherein the carrier material comprises a sheet mask and the infused material comprises a coating applied to the sheet mask.

9. The patient treatment of claim 1, wherein the carrier material comprises an adhesive portion that adheres to the skin.

10. The patient treatment of claim 9, further comprising a pattern of perforations configured to increase an elasticity between the adhesive portion and the infused material.

11. The patient treatment of claim 1, wherein the carrier material comprises one of silicone, Silagen®, a sheet mask, a polymer, a band-aid, a wound dressing, a gel, a steri-strip, SYLKE®, or Mepelex.

12. The patient treatment of claim 11, wherein the infused material comprises at least a portion of the carrier material.

13. The patient treatment of claim 11, wherein the infused material comprises a coating for the carrier material.

14. A patient-treatment article comprising:
a carrier material configured to be placed against skin of a patient, the carrier material comprising at least one of silicone, Silagen®, a sheet mask, a polymer, a band-aid, a wound dressing, a gel, a steri-strip, SYLKE®, or Mepelex;
an infused material disposed on or within at least a portion of the carrier material, the infused material comprising a skin-treatment component including arnica and bromelain and further comprising silver;
wherein the infused material is exposed at a surface of the carrier material such that, when the carrier material is placed against the skin of the patient, the infused material contacts the skin; and
wherein the carrier material further comprises an adhesive portion that adheres to the skin and a pattern of perforations between the adhesive portion and the infused material that provides elasticity.

15. A method of making a patient-treatment article, comprising:
providing a blank of carrier material configured to be placed over skin of a patient; and
infusing a skin-treatment component material into at least a portion of the carrier material, wherein the skin-treatment component material is exposed at a surface of the carrier material such that the skin-treatment component material is configured to contact a patient skin when the patient-treatment article is applied.

16. The method of claim 15, wherein the skin-treatment component material comprises at least arnica and bromelain.

17. The method of claim 16, wherein the skin-treatment component material comprises silver.

18. The method of claim 15, wherein infusing the skin-treatment component material comprises coating the carrier material with the skin-treatment component material.

19. The method of claim 15, further comprising applying an adhesive to the carrier material.

20. The method of claim 19, further comprising forming a pattern of perforations in the adhesive, thereby increasing an elasticity of the carrier material.
